(19) United States
(12) Patent Application Publication
(10) Pub. No.: US 2021/0008547 A1
(43) Pub. Date: Jan. 14, 2021

(54) DNA CONCENTRATE DISPENSING

(71) Applicant: Hewlett-Packard Development Company, L.P., Spring, TX (US)

(72) Inventors: Hilary ELY, Corvallis, OR (US); Adam HIGGINS, Corvallis, OR (US); Christie DUDENHOEFER, Corvallis, OR (US); Jeffrey A. NIELSEN, Corvallis, OR (US)

(73) Assignee: Hewlett-Packard Development Company, L.P., Spring, TX (US)

(21) Appl. No.: 16/603,460

(22) PCT Filed: Sep. 15, 2017
(86) PCT No.: PCT/US2017/051893

§ 371 Date: Oct. 7, 2019

(51) Int. Cl. B01L 3/00; G01N 35/10; C12Q 1/6844

(52) U.S. Cl. CPC B01L 3/5085; G01N 2035/1034; C12Q 1/6844; G01N 35/1011

(57) ABSTRACT

Examples disclosed herein relate to a device. Examples include a region selection engine to determine a plurality of regions on a well plate, a number of wells in each region, and a location of each well in each region; and a dispense engine to determine a quantity of DNA concentrate under 1 microliter to dispense in each well of the plurality of regions on the well plate, and the dispense engine to control a fluid dispensing device to eject the quantity of DNA concentrate into each well of each region of the well plate. In examples, a quantity of DNA fragments in the DNA concentrate is unknown.

### BACKGROUND

Various types of devices may be used to perform biological and chemical testing. One type of biological test may be a nucleic acid test used to identify DNA. Nucleic acid tests are a tool for the amplification of individual molecules for purposes of identifying and counting individual DNA molecule sequences.

### BRIEF DESCRIPTION OF THE DRAWINGS

The following detailed description references the drawings, wherein:

FIG. 1 is a block diagram of an example device;

FIG. 2 is a block diagram of an example device;

FIGS. 3A-3C is a chart showing a representation of the effect of the number of empty wells measured on the expected probability for a fraction of wells that are empty when there is an average of 1 DNA fragment copy per well according to an example;

FIG. 4 is a chart showing a representation of a predicted fraction of wells that are empty as a function of number of DNA concentrate droplets delivered per well according to an example;

FIG. 5 is a flowchart of an example process;

FIGS. 6A-6B are flowcharts of example processes which may be incorporated into the flowchart of FIG. 5; and

FIG. 7 is a block diagram of an example device to perform digital nucleic acid testing.

Throughout the drawings, identical reference numbers designate similar, but not necessarily identical, elements. The figures are not necessarily to scale, and the size of some parts may be exaggerated to more clearly illustrate the example shown. Moreover the drawings provide examples and/or implementations consistent with the description; however, the description is not limited to the examples and/or implementations provided in the drawings.

### DETAILED DESCRIPTION

Nucleic acid tests or digital nucleic acid tests (hereinafter “DNAT”) refers to a number of tests to provide a mechanism for identification and amplification of individual nucleic acid fragments, such as DNA, mRNA, RNA, in a fluid, hereinafter referred to as a “DNA concentrate”. A DNA concentrate may include any type of nucleic acid, such as, DNA, RNA, mRNA. As used herein, “DNA” refers to any nucleic acid and/or fragment thereof, such as DNA, RNA, mRNA, etc.

In some examples, DNA concentrate may be added to another fluid to amplify and identify any DNA fragments present in the DNA concentrate. The fluid is referred to as a reaction mix or reaction fluids and includes the compounds needed to perform DNAT. In some examples, DNAT samples of DNA concentrate and reaction fluids may be distributed over multiple reaction volumes or wells at a mean concentration below approximately one molecule per well. Amplification of single molecules may be achieved in a minority of the wells, providing a readout of the original number of molecules in the distributed sample.

In examples, DNAT includes applying heat to the reaction volumes to enable the amplification of the DNA fragments by the reaction mix. In some examples, a well with a DNA fragment may be treated to fluoresce to allow for determination of which wells include amplified DNA. In some examples, DNAT may include isothermal methods, such as, loop mediated isothermal amplification, nucleic acid sequence based amplification, strand displacement amplification, multiple displacement amplification recombinase polymerase amplification (RPA), etc. In other examples, DNAT may include thermocycled methods such as polymerase chain reaction (PCR), reverse-transcriptase polymerase chain reaction (RT-PCR), etc.

Some example DNAT devices may include mechanisms by which to partition a sample containing a DNA concentrate and reaction fluids into up to tens of thousands or more reaction volumes. The process of partitioning the DNA concentrate and reaction fluids into reaction volumes to achieve distribution of some DNA in some wells is time consuming and increases with the number of partitions performed.

In some examples, DNAT based distribution may be based at least in part on statistical distributions, such as the Poisson distribution. In such examples, the average number of copies of target DNA per well (C) may be described in terms of the number of “empty” wells (i.e., wells that contain zero copies) as follows:

E=exp(−C)

In these examples, E is the observed fraction of wells that are empty. In many cases, C is unknown. The equation above may be used to determine C from the measured value of E. The exponential dependence between the fraction of empty wells and the average copies per well may make it difficult to reach a desired range for detection, which may be referred to as the “sweet spot” for detection. The desired range, i.e., the sweet spot, refers to a range of target DNA in a reaction volume which provides a statically significant number of empty wells. For example, when there is an average of one copy of target DNA per well, about 37% of the wells are expected to be empty. But if there are 10 copies per well, less than 0.01% of the wells are expected to be empty. In these examples, when less than 0.01% of the wells are expected to be empty, accurate determination of the value E may be less likely. In this case at least 10,000 wells are needed to detect an empty well (since only 1 in 10,000 wells is expected to be empty). In some examples, the “sweet spot” that provides the most accurate results is approximately 1 to approximately 3 DNA copy per well. In some examples, the “sweet spot” that provides the most accurate results may be approximately 1 DNA copy per well.

For some example DNAT measurements devices and processes, accuracy of the measurements may be increased by: (1) adjusting the target DNA concentration in the reaction mixture to hit the sweet spot; and/or (2) increasing the number of partitions. While some example DNAT devices and technology focus on increasing the number of partitions, examples described herein, facilitate increased DNAT accuracy using a relatively small number of partitions by adjusting the target DNA concentration to increase likelihood of achieving the desired range for detection, i.e., the “sweet spot.” Accordingly, examples provided herein may adjust target DNA concentration to thereby achieve the desired range.

In examples and throughout the specification, a “well plate” refers to a physical structure to house one or more reaction volumes, which are also referred to as “reaction wells” or simply “wells.” In some examples, a well plate may include a commercially available plate with a plurality of wells (e.g., a 384 well plate, a 1534 well plate, etc.), a polymeric sheet with pockets formed therein, a lab-on-chip device, a material to receive a reaction volume (e.g., a porous material), or any other type of structure to receive reaction volumes. In contrast, a well refers to a single physical structure or location on a material to receive a single reaction volume. In examples, a well may be a single well of a commercially available well plate, a single pocket in a polymeric sheet, a single region of a lab-on chip-device, and a single region of a material. In some examples, a lab-on-chip device may include channels and/or chambers which may act as a well. In some examples, a material may be a porous material with regions which may act as a well

To address the issues described herein, in examples, a device may determine regions of a well plate and a quantity of DNA concentrate to be dispensed in each region. At the time of dispensing, the concentration of DNA in the DNA concentrate is unknown. DNAT may be performed on the well plate to determine a concentration of DNA in the DNA concentrate. The well plate may include a reaction mix to react with the DNA concentrate during DNAT. In some examples, the well plate may be pre-loaded with the reaction mix. In other examples, the reaction mix may be dispensed into wells of the well plate before, concurrent with, or after the DNA concentrate is dispensed. DNAT may include applying heat to the well plate for a certain duration. In some examples, the reaction mix and the DNA concentrate of each well may react such that any well(s) containing amplified DNA fragments may fluoresce or change color. In some examples, an optical sensor, such as, a photodetector, a pyrometer, an infrared sensor, a spectrophotometer, etc., may detect whether a well has fluoresced or changed color to determine if the well has amplified DNA fragments. In other examples, electrochemical analysis of the well plate may be used to determine if a well contains an amplified DNA fragments.

As shown herein, example devices may comprise engines, where such engines may be any combination of hardware and programming to implement the functionalities of the respective engines. In some examples described herein, the combinations of hardware and programming may be implemented in a number of different ways. For example, the programming for the engines may be processor executable instructions stored on a non-transitory machine-readable storage medium and the hardware for the engines may include a processing resource to process and execute those instructions. A “processor” may be at least one of a central processing unit (CPU), a semiconductor-based microprocessor, a graphics processing unit (GPU), a field-programmable gate array (FPGA) to retrieve and execute instructions, other electronic circuitry suitable for the retrieval and execution of instructions stored on a machine-readable storage medium, or a combination thereof.

In some examples, a device implementing such engines may include the machine-readable storage medium storing the instructions and the processing resource to process the instructions, or the machine-readable storage medium may be separately stored and accessible by the system and the processing resource. In some examples, engines may be implemented in circuitry. Moreover, processing resources used to implement engines may comprise a processor (e.g., a CPU), an application specific integrated circuit (ASIC), a specialized controller, and/or other such types of logical components that may be implemented for data processing.

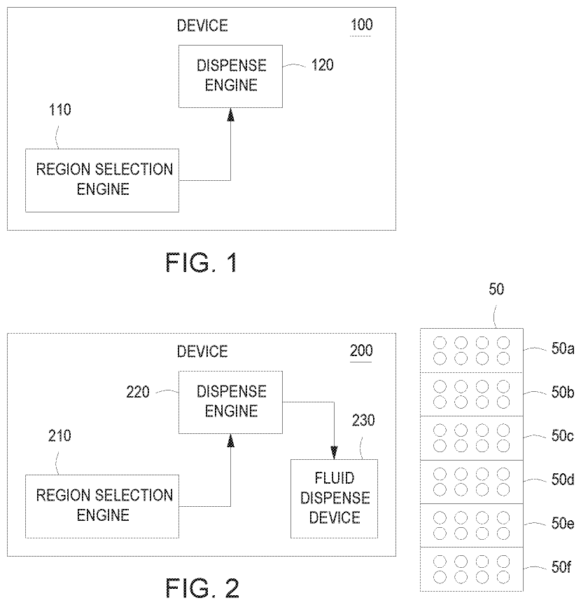

Turning now to the figures, and particularly to FIG. 1, this figure provides a block diagram that illustrates some components of an example device 100. Example device 100 may include a region selection engine 110 and a dispense engine 120. In these examples, device 100 may provide a method to adjust a target DNA concentrate for DNAT by ejecting DNA concentrate in the sweet spot in reaction wells.

In some examples, device 100 may be a device to control the ejection of a fluid. In some examples, device 100 may control ejection of a fluid by a fluid dispense device to dispense or eject a fluid. Example fluid dispense devices may include digital titration devices, pharmaceutical dispensation devices, lab-on-chip devices, fluidic diagnostic circuits, ink-based ejection devices, 3D printing devices, and/or other such devices in which amounts of fluids may be dispensed or ejected.

In examples, region selection engine 110 may determine a plurality of regions on a well plate, a number of wells in each region, and a location of each well in each region. In examples, DNAT may be performed on one well plate or across multiple well plates. In examples, region selection engine 110 may determine a number of regions on a well plate(s) to perform DNAT. In such examples, region selection engine 110 may determine the number of regions in response to a user input. In other examples, region selection engine 110 may determine the number of regions according to any characteristics of a DNA concentrate or reaction mix to be ejected/dispensed by device 100, e.g., DNA concentrate volume or reaction mix volume. In yet other examples, region selection engine 110 may determine the number of regions according to any characteristic of the well plate. In other examples, region selection engine 110 may determine the number of regions according to a characteristic of a biological or chemical test to be performed on the DNA concentrate. For example, in a lab-on-chip device, the number of regions may be determined according to the size of the lab-on-chip device or a type of test to be performed on the lab-on-chip device. In one such example, a lab-on-chip device may perform a test to identify a particular pathogen's DNA and it may be known that the DNA concentrate likely contains DNA with a particular concentration range thereby identifying the number of regions that may be used to identify the DNA concentration level through DNAT. In such an example, region selection engine 110 may determine the number of regions to perform DNAT to conserve the DNA concentrate for subsequent testing. In some examples, the number of regions may be greater than three (3). For example, the number of regions may be six (6) regions or seven (7) regions.

In examples, region selection engine 110 may determine a number of well(s) in each region. In examples, a region may include one or more wells. In examples, the number of wells in each region may be the same. In other examples, the number of wells in each region may differ. In examples, region selection engine 110 may determine the number of wells in each region in response to a user input. In other examples, region selection engine 110 may determine the number of wells in each region according to any characteristics of a DNA concentrate or reaction mix to be ejected by device 100, e.g., DNA concentrate volume. In yet other examples, region selection engine 110 may determine the number of wells in each region according to any characteristic of the well plate. For example, the number of wells in a region may be 48 wells for a well plate with 24 wells in a row or 96 wells for a well plate with 96 wells in a row. In another example, region selection engine 110 may determine the number of wells in each region according to a characteristic of a biological or chemical test to be performed on the DNA concentrate. In such an example, region selection engine 110 may determine the number of wells in a region to perform DNAT to conserve the DNA concentrate for subsequent testing.

In examples, region selection engine 110 may determine a location of each well in each region. In some examples, a well in each region may be adjacent to another well in the same region. In other examples, a well in each region may not be adjacent to another well in the same region.

In examples, dispense engine 120 may determine a quantity of DNA concentrate to dispense in each well of the plurality of regions on the well plate. In examples, dispense engine 120 may control a fluid dispense device to eject the quantity of DNA concentrate into each well of each region of the well plate. In examples, the quantity of DNA fragments in the DNA concentrate may be unknown. In examples, dispense engine 120 may determine the quantity of DNA concentrate to dispense/eject according to a user input. In other examples, dispense engine 120 may determine a quantity of DNA concentrate to dispense in each well according to any characteristics of a DNA concentrate or reaction mix, e.g., DNA concentrate volume or reaction mix volume. In yet other examples, dispense engine 120 may determine the quantity of DNA concentrate to dispense/eject in each well according to any characteristic of the well plate, e.g., well volume. In an example, dispense engine 120 may determine the quantity of DNA concentrate to dispense/eject in each well according to a characteristic of a biological or chemical test to be performed on the DNA concentrate. For example, in a lab-on-chip device, the quantity of DNA concentrate to dispense may be at a volume range of approximately 2 picoliters (pL) to approximately 1 microliter (μL) due to the size of the lab-on-chip device. Furthermore, the term “approximately” when used with regard to a value may correspond to a range of ±10%. In such an example, the lab-on-chip device may receive the DNA concentrate in a plurality of regions, channels, or wells formed in the lab-on-chip device. In examples, dispense engine 120 may determine the quantity of DNA concentrate to result in at least one region of the well plate receiving between 0.1 and 3 DNA fragments per well. In such examples, an amount or volume of DNA concentrate in each well of each such region may be between approximately 2 pL and approximately 200 μL.

In examples, the fluid dispense device may dispense/eject the quantity of DNA concentrate into each well of each region of the well plate. In examples, the fluid dispense device may include a fluid die with nozzles formed therein and an ejection chamber. In examples, nozzles may facilitate ejection/dispensation of a fluid. Fluid dispense devices may comprise fluid ejection actuators disposed proximate to the nozzles to cause fluid to be ejected/dispensed from a nozzle orifice. Some examples of types of fluid ejectors implemented in fluid dispense devices include thermal ejectors, piezoelectric ejectors, pressure pulse ejectors acoustic ejectors, syringes, pin transfer tools and/or other such ejectors that may cause fluid to eject/be dispensed from a nozzle. In some examples, the fluid dispense device may be removable. In one such example, the fluid die, nozzle, and ejection chamber of the fluid dispense device may be removable from the fluid dispense device. In examples, fluid dispense devices may be able to dispense fluids volumes from approximately 2 pL to approximately 200 μL. In such an example, the fluid dispense devices may dispense or eject a fluid drop with a drop volume between approximately 2 pL and approximately 300 pL per drop. In some such examples, fluid dispense devices may eject or dispense in a range from 1 to 200,000 drops of a fluid in a well. In examples, the range of fluid volumes that may be dispensed from fluid dispense devices may make it easier to deliver different quantities of DNA concentrate to each well.

Turning now to FIG. 2, this figure illustrates a diagram of an example of a fluid device 200. In the example of FIG. 2, a well plate 50 into which device 200 ejects or dispenses DNA concentrate is shown. The device 200 may include all features discussed with reference to the examples of FIG. 1. In examples, device 200 may include a region selection engine 210, a dispense engine 220, and a fluid dispense device 230.

In some examples, device 200 may be a device to control the ejection of a fluid. In some examples, device 200 may control ejection of a fluid by fluid dispense device 230 coupled thereto to dispense or eject a fluid. Example fluid dispense device 230 may include digital titration devices, pharmaceutical dispensation devices, lab-on-chip devices, fluidic diagnostic circuits, ink-based ejection devices, 3D printing devices, and/or other such devices in which amounts of fluids may be dispensed or ejected. In some examples, device 200 may be a fluid dispense device to dispense or eject a fluid into well plate 50. In such an example, device 200 may include a removable fluid ejector, such as a pipette or a fluid die with nozzles. In examples, well plate 50 may be coupled to device 200 to allow device 200 to dispense a fluid therein. In the following discussion and in the claims, the term “couple” or “couples” is intended to include suitable indirect and/or direct connections. Thus, if a first component is described as being coupled to a second component, that coupling may, for example, be: (1) through a direct electrical or mechanical connection, (2) through an indirect electrical or mechanical connection via other devices and connections, (3) through an optical electrical connection, (4) through a wireless electrical connection, and/or (5) another suitable coupling. In contrast, the term “connect,” “connects,” or “connected” is intended to include direct mechanical and/or electrical connections. In examples, well plate 50 may be coupled to a transportation mechanism to move or transport well plate 50 such that each well of well plate 50 may receive a fluid from ejection head 230. In other examples, well plate 50 may remain stationary and device 200 or a portion thereof, such as fluid dispense device 230, may travel or be transported such that each well of well plate 50 may receive a fluid from fluid dispense device 230. In yet further examples, both well plate 50 and device 200 may travel or move to allow each well in well plate 50 to receive a fluid from fluid dispense device 230.

In examples, region selection engine 210 may determine a plurality of regions on well plate 50, a number of wells in each region, and a location of each well in each region. Although described with respect to multiple regions on a single well plate, the examples are not limited thereto and each region may be disposed on a different well plate. In examples, region selection engine 210 may determine a number of regions on well plate 50 to perform DNAT. In the example of FIG. 2, six regions are depicted on well plate 50, in particular, region 50a, region 50b, region 50c, region 50d, region 50e, and region 50f. In such examples, region selection engine 210 may determine the number of regions in response to a user input. In other examples, region selection engine 210 may determine the number of regions according to any characteristics of a DNA concentrate or reaction mix to be ejected/dispensed by device 200, e.g., DNA concentrate volume or reaction mix volume. In yet other examples, region selection engine 110 may determine the number of regions according to any characteristic of the well plate. In another examples, region selection engine 110 may determine the number of regions according to a characteristic of a biological or chemical test to be performed on the DNA concentrate.

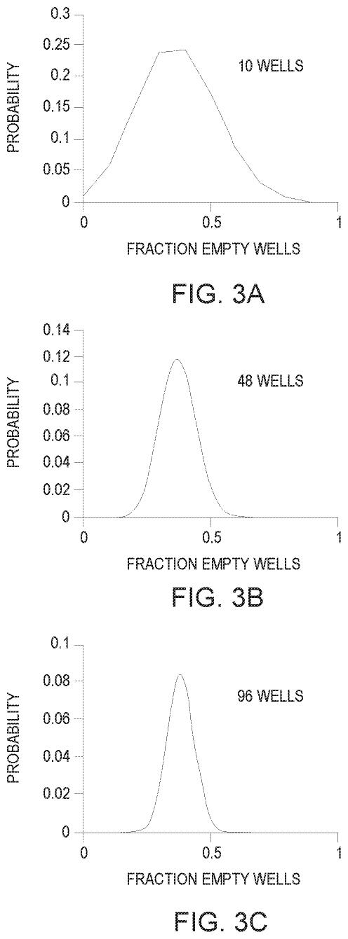

In some examples, another factor region selection engine 230 may considered is the effect of DNAT partitions on the expected confidence in the resulting DNA concentration measurement. While the Poisson distribution describes the expected statistical behavior at the population level, actual experimental measurements based on a finite number of partitions follow the binomial distribution. According to the binomial distribution, the greater the number of partitions, the more likely that the measured number of empty wells will match the expected value from the Poisson distribution. For example, if an average of 1 DNA copy per well is delivered, Poisson statistics tells us that we expect 37% of the wells to be empty. In an actual measurement, statistical variability may cause the measured empty well fraction to be distributed about this expected value. Referring now to FIGS. 3A-3C, FIG. 3A-3C are charts showing a representation of the effect of the number of empty wells measured on the expected probability for a fraction of wells that are empty when there is an average of 1 DNA fragment copy per well according to an example. As shown in FIGS. 3A-3C, the probability distribution for the empty well fraction becomes narrower as the number of wells increases. The chart in FIG. 3A shows the probability distribution for 10 wells, when there is an average of 1 DNA fragment copy per well. In the example of FIG. 3A, the probability distribution for the empty well fraction is the broadest. The chart in FIG. 3B shows the probability distribution for 48 wells, when there is an average of 1 DNA fragment copy per well. When there are 48 wells, there is a 95% confidence that the measured empty well fraction will be between 0.23 and 0.50. This corresponds to fragment copies per well (according to Eq. 1) ranging between 0.69 and 1.47. This is within 50% of the expected value of 1 fragment copy per well. As such, in some examples, the number of wells in a region may be 48 wells. In such an example, the number of wells in all six regions of well plate 50 may be 288 when all six regions have the same number of wells. Further in such examples, the use of less than 300 wells may allow for faster DNAT. The chart in FIG. 3C shows the probability distribution for 96 wells, when there is an average of 1 DNA fragment copy per well. In the example of FIG. 3C, the probability distribution for the empty well fraction is the narrowest.

Turning once again to FIG. 2, in examples, region selection engine 210 may determine a number of well(s) in each region. In examples, a region may include one or more wells. In examples, the number of wells in each region may be the same. In other examples, the number of wells in each region may differ. FIG. 2 depicts 8 wells in each of region 50a, region 50b, region 50c, region 50d, region 50e, and region 50f. In examples, region selection engine 210 may determine the number of wells in each region in response to a user input. In other examples, region selection engine 210 may determine the number of wells in each region according to any characteristics of a DNA concentrate or reaction mix to be ejected by device 200, e.g., DNA concentrate volume. In yet other examples, region selection engine 210 may determine the number of wells in each region according to any characteristic of the well plate. In another example, region selection engine 210 may determine the number of wells in each region according to a characteristic of a biological or chemical test to be performed on the DNA concentrate. In such an example, region selection engine 210 may determine the number of wells in a region to perform DNAT to conserve the DNA concentrate for subsequent testing. In examples, region selection engine 210 may determine a location of each well in each region. In some examples, as shown in FIG. 2, a well in each region may be adjacent to another well in the same region. In other examples, a well in each region may not be adjacent to another well in the same region.

In examples, dispense engine 220 may determine a quantity of DNA concentrate to dispense in each of well in each of region 50a, region 50b, region 50c, region 50d, region 50e, and region 50f on well plate 50. In examples, dispense engine 220 may control fluid dispense device 230 to eject the quantity of DNA concentrate into each of the plurality of wells in each of region 50a, region 50b, region 50c, region 50d, region 50e, and region 50f on well plate 50. In examples, the quantity of DNA fragments in the DNA concentrate may be unknown. In examples, dispense engine 220 may determine the quantity of DNA concentrate to dispense/eject according to a user input. In other examples, dispense engine 120 may determine a quantity of DNA concentrate to dispense in each well according to any characteristics of a DNA concentrate or reaction mix, e.g., DNA concentrate volume or reaction mix volume. In yet other examples, dispense engine 220 may determine the quantity of DNA concentrate to dispense/eject in each well according to any characteristic of the well plate, e.g., well volume. In an example, dispense engine 220 may determine the quantity of DNA concentrate to dispense/eject in each well according to a characteristic of a biological or chemical test to be performed on the DNA concentrate. For example, in a lab-on-chip device, the quantity of DNA concentrate to dispense may be at a volume range of approximately 2 pL to approximately 1 μL due to the size of the lab-on-chip device. In such an example, the lab-on-chip device may receive the DNA concentrate in a plurality of regions, channels, or wells formed in the lab-on-chip device. In examples, dispense engine 220 may determine the quantity of DNA concentrate to result in at least one of region 50a, region 50b, region 50c, region 50d, region 50e, and region 50f receiving between 0.1 and 3 DNA fragments per well. In such examples, an amount or volume of DNA concentrate in each well of region 50a, region 50b, region 50c, region 50d, region 50e, and region 50f may be between approximately 2 pL and approximately 200 μL.

In examples, fluid dispense device 230 may dispense/eject the quantity of DNA concentrate into each well in each of region 50a, region 50b, region 50c, region 50d, region 50e, and region 50f of well plate 50. In examples, fluid dispense device 230 may include a fluid die with nozzles formed therein and an ejection chamber. In examples, nozzles may facilitate ejection/dispensation of a fluid. Fluid dispense device 230 may comprise fluid ejection actuators disposed proximate to the nozzles to cause fluid to be ejected/dispensed from a nozzle orifice. Some examples of types of fluid ejectors implemented in fluid dispense devices include thermal ejectors, piezoelectric ejectors, pressure pulse ejectors acoustic ejectors, syringes, pin transfer tools and/or other such ejectors that may cause fluid to eject/be dispensed from a nozzle orifice. In examples, dispensing may include generating a control pulse to electrically actuate the fluid actuator of fluid dispense device 230 to thereby dispense the DNA concentrate. In examples, fluid dispense devices 230 may be able to dispense fluids volumes between approximately 2 pL to approximately 200 μL. In such an example, fluid dispense device 230 may dispense or eject a fluid drop with a drop volume between approximately 2 pL and approximately 300 pL per drop. In some such examples, fluid dispense device 230 may eject or dispense in a range from 1 to 200,000 drops of a fluid in each well of well plate 50. In examples, the range of fluid volumes that may be dispensed from fluid dispense device 230 may make it easier to deliver different quantities of DNA concentrate to each well.

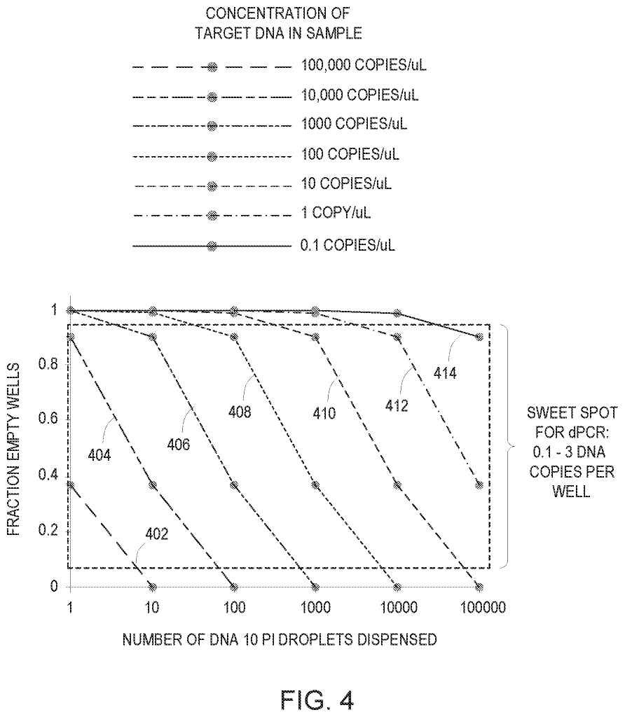

Referring now to FIG. 4, FIG. 4 is a graphical representation of an expected fraction of empty wells as a function of the number of droplets dispensed per well according to an example. Curve 402 shows DNA concentrate 100,000 copies of DNA per 1 μL. Curve 404 shows DNA concentrate 10,000 copies of DNA per 1 μL. Curve 406 shows DNA concentrate 1,000 copies of DNA per 1 μL. Curve 408 shows DNA concentrate 100 copies of DNA per 1 μL. Curve 410 shows DNA concentrate 10 copies of DNA per 1 μL. Curve 412 shows DNA concentrate 1 copies of DNA per 1 μL. Curve 414 shows DNA concentrate 0.1 copies of DNA per 1 μL. As shown, a good range for DNAT measurement of DNA concentration is from about 0.1 DNA copies per well to about 3 copies per well. Delivery of a different number of DNA concentrate droplets to each region of the well plate ensures that there will be a region that will be in this DNAT “sweet spot.” For example, when the target DNA concentration is 100,000 copies/uL (curve 402), delivery of a single 10 pL drop would result in an average of 1 copy per well, which is in the sweet spot for DNAT. On the other hand, for a million fold lower DNA concentration of 0.1 copies/uL (curve 410) about 100,000 droplets would need to be delivered to each well to reach the sweet spot. FIG. 4 demonstrates that it may be feasible to accurately measure DNA concentration by DNAT over a very wide range of DNA concentrations spanning 7 orders of magnitude. This capability may be enabled by fluid dispense device 230, which may make it possible to precisely and conveniently dispense a different number of DNA concentrate droplets into each region of well plate 50. In the example described with respect to FIG. 4, 576 wells are used (e.g., less than half of a commercially available 1536 well plate).

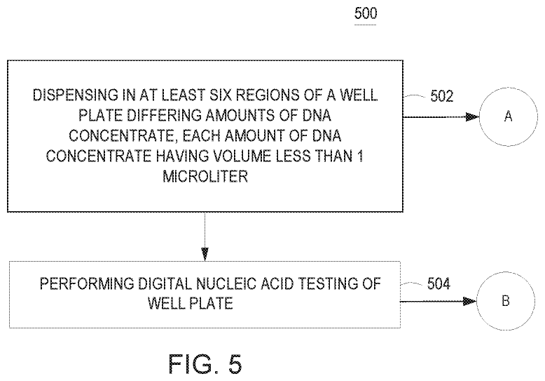

Turning to FIG. 5, this figure provides a flowchart 500 that illustrates a sequence of operations corresponding to a process to perform DNAT. As shown in 5, with a processor, differing amounts of DNA concentrate may be dispensed in each well of at least six region of a well plate at block 502. In the example of FIG. 5, each amount of DNA concentrate dispensed may have a volume less than 1 microliter (1 μL). In examples, dispensing in each well of at least six region of a well plate differing amount of DNA concentrate may include generating a control pulse to electrically actuate a fluid actuator of a fluid dispense device to thereby dispense the first amount of DNA. In the example of FIG. 5, the first amount of DNA concentrate may be in a range of approximately 2 pL to approximately 200 μL. In examples, one or more wells may be in each region.

At 504, DNAT may be performed of the well plate. In the example of FIG. 5, the concentration of DNA in the DNA concentrate is unknown when dispensing.

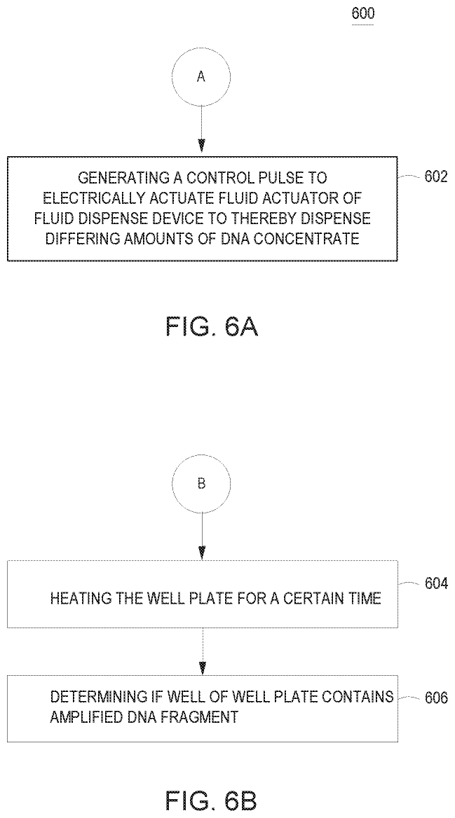

Turning now to FIGS. 6A-6B, FIGS. 6A-6B are flowcharts of example processes which may be incorporated into the flowchart of FIG. 5.

At 602, dispensing in each well of at least six region of a well plate differing amount of DNA concentrate may include generating a control pulse to electrically actuate a fluid actuator of a fluid dispense device to thereby dispense the differing amounts of DNA.

At 604, performing DNAT on a well plate may include heating the well plate for a duration time.

At 606, performing DNAT on a well plate may include determining if a well of the well plate contains amplified DNA fragments. In some examples, an optical sensor may detect whether a well has fluoresced to determine if the well has amplified DNA fragments.

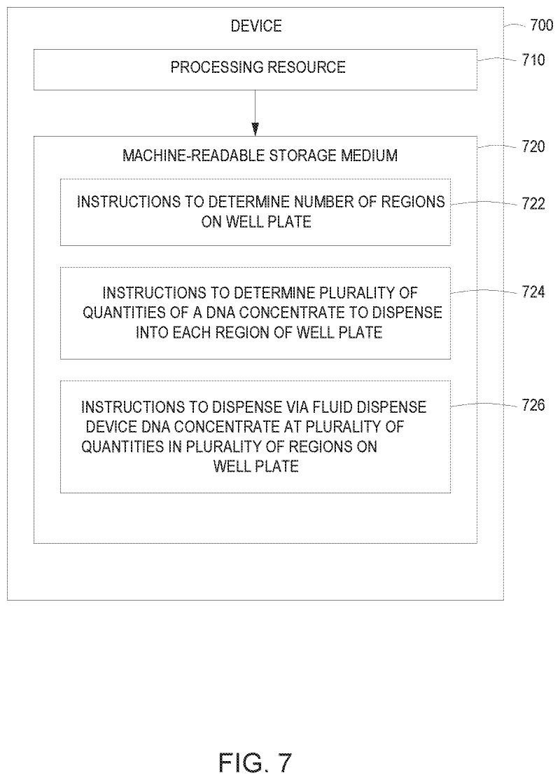

Turning now to FIG. 7, FIG. 7 is a block diagram of an example device to perform digital nucleic acid testing. In the example of FIG. 7, device 700 includes a processing resource 710 and a machine-readable storage medium 720 comprising (e.g., encoded with) instructions 722, 724, and 726 executable by processing resource 710. In some examples, storage medium 720 may include additional instructions. In some examples, instructions 722, 724, 726, and any other instructions described herein in relation to storage medium 720, may be stored on a machine-readable storage medium remote from but accessible to device 700 and processing resource 710 (e.g., via a computer network). In some examples, instructions 722, 724, and 726 may be instructions of a computer program, computer application (“app”), agent, or the like, of device 700. In other examples, the functionalities described herein in relation to instructions 722, 724, and 726 may be implemented as engines comprising any combination of hardware and programming to implement the functionalities of the engines, as described above.

In the example of FIG. 7, instruction 722 may determine a number of regions on a well plate. In examples, the number of regions may be determined according to a user input. In other examples, the number of regions may be determined according to any characteristic of a fluid to be dispensed, such as a DNA concentrate, a reaction mix, or any other fluid (e.g., a fluid volume). In yet other example, the number of regions may be determined according to any characteristic of a well plate(s) (e.g., a number of wells) to receive the fluid. In examples, one or more wells may be disposed in each region. As described above with respect to FIG. 3B, in some examples, the number of wells in a region may be 48 to achieve good DNAT accuracy. In some examples, the number of wells in each region may be the same. For example, if each region includes 48 wells, a total of 288 wells may be used in a DNAT process with six (6) regions. In other examples, the number of wells in each region may differ. In some examples, three (3) or more regions may be determined on a well plate. In such an example, the well plate may be a material or a lab-on-chip device.

In instructions 724, a plurality of quantities of a DNA concentrate to dispense into each region of the well plate may be determined. In examples, the quantities of a DNA concentrate may be determined according to a user input. In other examples, the quantities of a DNA concentrate may be determined according to any characteristic of a fluid to be dispensed, such as a DNA concentrate, a reaction mix, or any other fluid. In another example, the quantities of a DNA concentrate may be determined according to any characteristic of a well plate(s) to receive the fluid (e.g., a number of wells). In yet another example, the quantities of a DNA concentrate may be determined according to a characteristic of a biological or chemical test to be performed on the DNA concentrate.

In instruction 726, a fluid dispense device may dispense DNA concentrate at the plurality of quantities in the plurality of regions on the well plate. In examples, the quantity of DNA concentrate dispensed in each well of each region may be the same. In examples, the fluid dispense device may be any type of device to dispense or eject a fluid. In some examples, the fluid dispense device may include a fluid die and ejection chamber(s). In such examples, the fluid dispense device may eject a fluid, such as DNA concentrate, in droplets with a volume range of approximately 2 pL to approximately 300 pL. In such examples, an amount of fluid dispensed in each well of a region may have a volume in a range of approximately 2 pL to approximately 1 μL.

In examples, DNAT may be performed on the well plate. In such examples, the well plate may be heated for a duration of time. In examples, the well plate may be heated to a constant temperature for the duration of time. In other examples, the well plate may be heated to varying temperatures for the duration of time.

In examples, DNAT may include determining the number of regions containing amplified DNA fragments. In such examples, a reaction mix may react with DNA fragments to fluoresce or change color. In such an example, an optical sensor may detect the fluorescence of an amplified DNA fragment in a well. In the example of a lab-on-chip device or material, the optical sensor may be disposed on the material or part of the lab-on-chip device. In other examples of a lab-on-chip device or material, the optical sensor may be coupled to the material or lab-on-chip device to detect amplified DNA fragments. In other examples, electrochemical analysis of the well plate may be used to determine if a well contains an amplified DNA fragments.

In some examples, instructions 722, 724, and 726 may be part of an installation package that, when installed, may be executed by processing resource 710 to implement the functionalities described herein in relation to instructions 722, 724, and 726. In such examples, storage medium 720 may be a portable medium, such as a CD, DVD, flash drive, or a memory maintained by an imaging device from which the installation package can be downloaded and installed. In other examples, instructions 722, 724, and 726 may be part of an application, applications, or component already installed on imaging device 700 including processing resource 710. In such examples, the storage medium 720 may include memory such as a hard drive, solid state drive, or the like. In some examples, functionalities described herein in relation to FIG. 7 may be provided in combination with functionalities described herein in relation to any of FIGS. 1-6.

What is claimed is:

1. A device, comprising:
a region selection engine to determine a plurality of regions on a well plate, a number of wells in each region, and a location of each well in each region; and
a dispense engine to determine a quantity of DNA concentrate under 1 μL to dispense in each well of the plurality of regions on the well plate, and the dispense engine to control a fluid dispense device to eject the quantity of DNA concentrate into each well of each region of the well plate, a quantity of DNA fragments in the DNA concentrate is unknown,
wherein at least one region of the plurality of regions is to receive at least between 0.1 and 3 DNA fragments per well.

2. The device of claim 1, wherein the fluid dispense device is a fluid die with an ejection chamber.

3. The device of claim 2, wherein the fluid dispense device is removable.

4. The device of claim 1, wherein the well plate is a lab-on-chip device.

5. The device of claim 1, wherein the region selection engine determines three (3) or more regions on a well plate.

6. The device of claim 1, wherein the well plate is a material.

7. The device of claim 1, wherein the fluid dispensing device ejects DNA concentrate in droplets with a volume range of approximately 2 pL-approximately 300 pL.

8. The device of claim 1, wherein the number of wells in each region is the same.

9. The device of claim 1, wherein the number of wells in each region differ.

10. A method for digital nucleic acid testing, comprising:
dispensing, with a processor, in at least six regions of a well plate differing amounts of DNA concentrate, each amount of DNA concentrate having a volume less than 1 μL; and
performing, by a processor, digital nucleic acid testing of the well plate, wherein the concentration of DNA in the DNA concentrate is unknown.

11. The method of claim 10, wherein the first amount, the second amount, the third amount, the fourth amount, the fifth amount, and the sixth amount are in a range of approximately 2 pL to approximately 1 μL.

12. The method of claim 10, wherein dispensing in at least six regions of a well plate differing amounts of DNA concentrate comprises generating a control pulse to electrically actuate a fluid actuator of a fluid dispense device to thereby dispense the differing amounts of DNA concentrate.

13. The method of claim 10, wherein performing digital nucleic acid testing of the well plate comprises:
heating the well plate for a duration of time; and
determining if a region of the well plate contains amplified DNA fragments.

14. A non-transitory machine-readable storage medium comprising instructions executable by a processing resource to:
determine a number of regions on a well plate;
determine plurality of quantities of a DNA concentrate to dispense into each region of the well plate; and
dispense via a fluid dispensing device a DNA concentrate at the plurality of quantities in the plurality of regions on the well plate, the fluid dispensing device to dispense DNA concentrate in volumes ranging from approximately 2 pL to approximately 1 μL.

15. The medium of claim 14, wherein the number of regions on a well plate is three (3) or more.
